(19) United States
(12) Patent Application Publication
(10) Pub. No.: US 2021/0127909 A1
(43) Pub. Date: May 6, 2021

(54) CORELESS ROLL OF ABSORBENT TISSUE WEB

(71) Applicant: Essity Hygiene and Health Aktiebolag, Goteborg (SE)

(72) Inventors: Yves Stoeffler, St. Ouen (FR); Ali Bagherzadeh, Goteborg (SE); Mattias Bengtsson, Goteborg (SE)

(21) Appl. No.: 16/491,602

(22) PCT Filed: Mar. 10, 2017
(86) PCT No.: PCT/EP2017/055747

§ 371 Date: Sep. 6, 2019

(51) Int. Cl. A47K 10/16; B65H 18/28

(52) U.S. Cl. CPC A47K 10/16; B65H 2701/1924; B65H 2515/12; B65H 18/28

(57) ABSTRACT

A coreless roll of absorbent tissue web is provided, the web having a length and a width, and the roll defining a central axial hole that extends from a first end of the roll to a second end of the roll in a direction perpendicular to the length of the absorbent tissue web. The coreless roll has a high density. The absorbent tissue web includes an outer perforated length located in the periphery of the coreless roll, the outer perforated length having a plurality of perforation lines across the width of the absorbent tissue web such as to form individual sheets. The absorbent tissue web of the coreless roll further includes an inner unperforated length of the absorbent tissue web.

### CROSS-REFERENCE TO RELATED APPLICATION

This application is a national phase entry of, and claims priority to, International Application No. PCT/EP2017/055747, filed Mar. 10, 2017, with the same title as listed above. The above-mentioned patent application is incorporated herein by reference in its entirety.

### TECHNICAL FIELD

This application relates to a coreless roll of absorbent tissue web, the absorbent tissue web having a length and a width, the roll defining a central axial hole that extends from a first end of the roll to a second end of the roll in a direction perpendicular to the length of the absorbent tissue web.

### BACKGROUND

Most rolled products, such as bath tissue and paper towels, are made with cores, which serve a base upon which the product web is wound during manufacturing. Moreover, the core enables the rolled product to be positioned in a dispenser by a consumer. The core is often a relatively rigid cylindrical cardboard core which provides stability to the inner windings of the rolled product. For example, rolls of bath tissue or paper towels consist of a continuous length of absorbent material, divided into individual product “sheets” separated from each other by lines of perforations. The product rolls are typically mounted on a spindle for dispensing. These conventional rolls have the disadvantages of material cost for the core, adhesive etc. In addition, the core provides quite a large void in the center of the roll which is unused, i.e. does not comprise web material. The rolls with cores are thus commonly quite large in diameter if they comprise a long length of webs.

To overcome at least some of the disadvantages with conventional rolled products, coreless rolls have been produced. A coreless roll is one which does not have a separate, relatively rigid, independent, non-tissue core component such as the one described above. Coreless rolls eliminate the cost of core materials and the associated glue, and the coreless rolls generate no waste. Comparably they also comprise more web material than a cored roll of the same diameter since they are more narrowly wound at the center. However, the lack of stabilizing means in the form of a core renders the inner windings of a coreless roll unstable, and they may collapse. Collapsing typically occurs in the manufacture process of coreless rolls when the temporary mandrel is extracted after completing the winding, when the log is sawed into coreless rolls, or during storage and transport of the finished product. A result of collapsing is more difficult mounting to the spindle of the dispenser, and it effects the sense of quality that the consumer perceives. In order to overcome the issue, many coreless rolls are stabilized at the windings closest to the center of the roll, e.g. by addition of water or starch, to stiffen the absorbent tissue web so they can maintain the shape of the hole that is necessary for the consumer to be able to easily slide the roll onto the spindle of the dispenser prior to use. However, the stabilization renders the inner windings of the roll stiff and may cause undesired premature breaking and forming of a non-useable stiff “cigar” of inner windings.

Thus, it would be desirable to provide a coreless roll that addresses these various issues with conventional designs. For example, it would be desirable to provide a coreless roll in which the entire length of paper can be easily dispensed from the roll.

### SUMMARY

To address these and other problems with conventional designs, the present invention proposes a coreless roll with the technical features described below.

In one embodiment, a coreless roll of absorbent tissue web is provided, the web having a length and a width, the roll defining a central axial hole that extends from a first end of the roll to a second end of the roll in a direction perpendicular to the length of the web. The coreless roll has a high density, and the absorbent tissue web of the coreless roll comprises an outer perforated length of absorbent tissue web arranged in the periphery of the roll and the outer perforated length comprising a plurality of perforation lines across the width of the web such as to form individual sheets. The absorbent tissue web of the coreless roll includes an inner unperforated length of absorbent tissue web.

In one embodiment, the outer perforated length of the absorbent tissue web is arranged at an outer part of the coreless roll, wherein the outer part of the coreless roll is arranged in the periphery of the coreless roll. The inner unperforated length of absorbent tissue web is arranged at the inner part of the coreless roll close to the central axial cavity. The unperforated length of the web is hence arranged closer to the central axial hole than the outer perforated length of the web.

In another embodiment, the perforation lines of the perforated length extend substantially across the entire width of the web, or at least over a majority of the width. A perforation comprises alternating perforated segments and unperforated segments, of even or uneven distribution across the width. The absorbent tissue web is intended to break at the perforation lines at dispensing to define the size of the individual sheets. The term “perforation lines” as used herein should cover any intermittent or continuous weakening where the remaining strength of the web ranges between 25% and 70%, preferably 30% to 65%.

To this end, perforation lines are distributed along the absorbent tissue web extending from the outer transverse edge of the web to a set distance from the inner transverse edge. The inner unperforated length of the absorbent tissue web is one continuous length of material without perforation lines. Hence, the inner unperforated length of the absorbent tissue web may be dispensed as one single piece, i.e. a long “individual sheet”. The lack of perforation lines eliminates or at least greatly reduces the potential breaking points which perforation lines give rise to. Tears are often initiated at the point where the perforation lines meet the longitudinal edge of the web.

In a further embodiment, a high density coreless roll is herein defined as a coreless roll with a density of at least 235 kg/m³. The absorbent tissue web of such a roll is densely packed i.e. compressed radially, and the densely, tightly wound inner windings thereby stabilize the central axial hole so as to maintain the shape of the central axial hole. However, densely packed inner windings, which are wound at a small radius, also adhere more easily to each other. The inner windings of a high density coreless roll are therefore prone to cause premature tearing at the perforation lines and formation of a cigar. This is avoided by implementing an inner unperforated length according to some embodiments of the invention.

The density of the roll may be determined by conventional techniques. For example, the density of the roll can be calculated from the dimensions of the roll and the weight of the roll, both which can be easily measured.

In one embodiment, only the inner part of the coreless roll is of high density, i.e. the outer part of the roll is more loosely packed and the inner part is more densely packed.

In yet another embodiment, the coreless roll may include absorbent tissue material wherein the absorbent tissue material may be toilet tissue paper, hand towel tissue paper or kitchen towel tissue paper. The coreless roll may be a toilet paper roll, hand towel paper roll or kitchen towel paper roll.

In a further embodiment, the inner unperforated length of the absorbent tissue web arranged close to the central axial hole provides increased tear strength to the inner windings of the coreless roll. It therefore counters the increased friction at the innermost windings, which results in consistent unwinding and dispensing all the way through all the windings of the coreless roll.

According to some embodiments of the invention, the consumer may unwind and dispense the entire inner unperforated length of the absorbent tissue web and thus there is no waste.

In one embodiment, the unperforated length of the absorbent tissue web may be longer than the length of the individual sheets.

In another embodiment, the unperforated length of the absorbent tissue web may have a length of 1 to 6 meters, preferably 2 to 5 meters and most preferably 2 to 3 meters.

In a further embodiment, the unperforated length of the absorbent tissue web may be arranged within the innermost 10 meters of the absorbent tissue web, preferably within the innermost 6 meters of the absorbent tissue web. The innermost meters of the absorbent tissue web are defined as starting at the central axial hole of the coreless roll and winding outwards towards the periphery of the coreless roll.

In yet another embodiment, the unperforated length of absorbent tissue web may represent the innermost windings arranged at the inner radius of the coreless roll such as to define the central axial hole. In such a case, the unperforated length of absorbent tissue web defines the last piece of the roll, i.e. the last piece of web dispensed.

In one embodiment, the innermost length of the absorbent tissue web closest to the central axial hole may according to some examples comprise perforation lines, for example so that there are 1-4 individual sheets of conventional size lining the central axial hole. The unperforated length of web is in such case arranged following that perforated innermost length.

In another embodiment, the coreless roll may include an inner portion arranged closest to the central axial hole, wherein the windings of absorbent tissue web of the inner portion are releasably attached to each other. The attachment is present between the windings of the web and does not affect the ply bonding of the web. The attachment is detectable with a tensile tester. The tensile strength needed to detach the attached sheets is higher than the respective non-attached webs.

In a further embodiment, the inner portion may circumvent the central axial hole and is the absorbent tissue web at the innermost radial part of the coreless roll. The inner portion extends along the entire width of the web in axial direction and outwardly in radial direction from the central axial hole. It may extend about up to 2 cm in radial direction from the center of the central axial hole. The axial direction is defined as the direction in which the central axial hole extends, i.e. along the width of the absorbent tissue web of the coreless roll.

In yet another embodiment, the dense packing of the windings of the inner portion cause the adjacent windings of the inner portion to bond to each other. The bonding between the windings of the inner portion may also be achieved by bonding means.

The attached inner windings contribute to stabilizing the inner portion such as to maintain the shape of the central axial hole. This is desirable to ensure proper loading of the coreless roll, and to ensure proper dispensing by maintaining the shape of the central hole until the entire web is finished.

In one embodiment, the coreless roll has an inner portion closest to the central axial hole and an outer portion at the periphery of the roll. The portions are defined in radial direction, i.e. the outer portion consists of windings wounds closer to the periphery of the coreless roll than the windings of the inner portion. The inner portion is arranged inside the outer portion.

In some embodiments, there are no or little attachment between the windings of the outer portion. The attachment force between the windings of the inner portion is therefore higher than the attachment force between the windings of the outer portion.

In another embodiment, the inner portion may include the inner unperforated length of the absorbent tissue web. The inner portion may be radially larger, i.e. comprise more windings, than the portion constituted by the unperforated length of the web. The benefit of improved dispensing from the innermost part of the coreless roll is still achieved at least partly when the innermost part of the inner portion is made up of a continuous non-perforated web length.

In a further embodiment, the inner portion may correspond to the inner unperforated length of the absorbent tissue web, i.e. the length of web material that constitute the inner unperforated length corresponds to the length of web material that constitutes the windings of the inner portion.

The inner portion of the coreless roll may be stiffened so as to maintain the shape of the central axial hole.

In yet another embodiment, the highly densely packed windings of the inner portion provide the stiffness to inner portion, thereby contributing to maintaining the shape of the central axial hole. The inner portion may also be further stiffened.

In one embodiment, the inner unperforated length of the absorbent tissue web contributes to improved dispensing compared to coreless rolls that are provided with perforation lines at its entire length. Such commonly known coreless rolls easily break prematurely at the perforation lines inside the dispenser when the stiffened inner portion is reached. The creation of a stiffened “core” at the center of the coreless roll, which is difficult to dispense, is sometimes referred to as a cigar. The creation of paper cigars is avoided with the present invention.

A stiffened inner portion does not collapse at the lengthwise edges of the web at the central axial hole at either the first or second end surface. Moreover, the inner tail at the central axial hole does not collapse. The geometry of the hole is maintained, i.e. the geometry of the winding mandrel is perceivable. This is not the case with a non-stiffened inner portion.

In one embodiment, the length of the unperforated part of the absorbent tissue web corresponds at least partly to the length of the web defining the stiffened inner portion, so that the increased tensile strength in the web of the unperforated part, as compared to the perforated part, corresponds to the increased tear strength of the stiffened inner portion. A better dispensing of the inner part of the roll is thereby achieved. The innermost meters represent the web length which might cause the “cigar” due to the stiffening of the inner windings, and the inner windings attachment to each other. This is avoided by ceasing to perforated the last 1 to 6 meters of the web on the coreless roll. The unperforated length could be up to 10 meters of the entire length of the web on the coreless roll, but that renders a large portion of the roll unperforated. It may be undesirable by the consumer.

In another embodiment, stabilizing means may stiffen the inner windings of the absorbent tissue web closest to the central axial hole so as to maintain the shape of the central axial hole. The stiffening and stabilizing of the central part of the coreless roll may be achieved by e.g. water spray, addition of resins, winding on a star-shaped mandrel etc. These stabilizing means cause the inner windings of the inner portion to adhere to each other and form a very form-stable central axial hole. The form-stable central axial hole reduces the risk of the central axial hole collapsing. A collapsed central axial hole renders it more difficult to mount the coreless roll on a spindle in a dispenser or on an adapter for mounting in a dispenser. Thereby, a form-stable central axial hole achieves improved loading of the roll to a spindle of a dispenser.

During dispensing of absorbent tissue web from the inner portion, the inner windings might be so tightly wound and adhered to each other that cigars are formed. Cigars are formed by unintentional premature tearing in the stiffened inner portion of the roll. The unperforated length of web minimizes such risks because it strengthens the length of web. It thereby effects the dispensability and the possibility to use the end of the roll.

In a further embodiment, the stiffened inner portion may be stiffer than the outer portion arranged closest to the periphery of the roll. In such an example the absorbent tissue web of the stiffened inner portion may be stiffer than the absorbent tissue web of the outer portion.

In yet another embodiment, the inner portion may be stiffened by application of a composition to the absorbent tissue web wound closest to the central axial hole. The composition may comprise water, starch, plasticizers, strengthening resins, adhesives etc alone or in combination.

In one embodiment, the inner portion may be further stiffened by application of water applied to the absorbent tissue web wound closest to the central axial hole. In other words, the windings of the inner portion are further attached by applying water to the web material during manufacturing. The paper absorbs water and the water migrates in radial direction. The final water coverage constitutes the stiffened inner portion of the coreless roll. The water evaporates but leaves the web stiffer than prior to the water addition, and the water effected windings attached to each other.

In another embodiment, the water may be applied through water spraying a length of web corresponding to the inner portion before winding the web to a coreless roll. In one example, water is added to the innermost 2 meters of the coreless roll. In one example the water is added to a length of absorbent material corresponding to the inner unperforated length. The water may also be applied to the inner but not innermost of the windings. Due to the properties of the water is may migrate to adjacent windings depending amongst other things on the paper quality and the amount of water added.

In a further embodiment, the inner portion may be stiffened by application of adhesive applied to the absorbent tissue web wound closest to the central axial hole. Adhesives also contribute to the attachment between the windings of the inner portion.

In yet another embodiment, the inner portion may be stiffened by application of starch applied to the absorbent tissue web wound closest to the central axial hole. The starch may be mixed with water and applied as a water spray.

In one embodiment, the unperforated length of absorbent tissue web may represent inner windings arranged at the inner radius of the coreless roll. In one example the inner unperforated length is the innermost windings and in one example the inner unperforated length is not the innermost windings, but arranged adjacent the innermost windings.

In some embodiments, there are perforation lines present at the innermost windings and the inner unperforated length is arranged between the outer perforated length of web and the innermost perforated length of web. The innermost length of web may for example be the length extending from the inner transverse edge of the web to about 1 meter from the same transverse edge of the web.

In one embodiment, the first and/or second end of the coreless roll may include an end indentation at the inner portion extending in axial direction and arranged around the central axial hole.

In another embodiment, the end indentation around the central axial hole may be a permanently compressed portion of the roll. The indentation may be cone shaped, cylinder shaped or any other suitable shape. The indentation may cover a radius of the coreless roll made up of up to 12 meters of absorbent tissue web. In such a case the inner unperforated length may correspond to the entire length making up the indentation, or correspond to part of that length.

In a further embodiment, the first and/or second end of the coreless roll may have a shape of revolution with a cross section decreasing towards the central axial hole of the roll, i.e. cone shaped. The decrease towards the central axial hole may start about 2 cm in radial direction from the center of the central axial hole, or even closer to the central axial hole. The innermost windings closest to the central axial hole are, if the indentation is cone shaped, the windings most effected by the compression that give rise to the indentation.

In yet another embodiment, the coreless roll may include inner indentations at the inner surface of the central axial hole. Such indentations may be achieved by winding the absorbent tissue web to a mandrel with a pattern e.g. star shaped or provided with outwards directed retractable pins. Once the web of material is completely wound on the mandrel the mandrel is retracted to create the central axial hole in the coreless roll. The central axial hole maintains the shape of the mandrel.

Different elements for stabilizing the inner portion of the coreless roll may be combined, e.g. water spray, starch, plasticizer, strengthening resins, end indentations and a star shaped mandrel during manufacturing.

In one embodiment, the outer perforated length of the absorbent tissue web may include a plurality of perforation lines across the width of the web at a distance of less than 40 cm from each other, such as to form individual sheets of less than 40 cm length. The length is chosen depending on the desired individual sheet length. The individual sheets of the perforated length may be of equal of the same length. The individual sheets may have a length in the range of between 8 cm and 40 cm. A common size of individual sheet lengths on a toilet paper coreless roll is 12.5 cm.

In another embodiment, a coreless roll with a stiffened inner portion, circumventing the central axial hole, reduces the risk of the windings surrounding the hole collapsing. To the contrary, these inner windings surrounding the hole are firm and uphold the shape and form of the central axial hole. The stiffened inner portion thus provides a visible hole that the user can easily identify. It makes the mounting of the coreless roll to a spindle easier. The stiffened inner portion hence ensures good loading of the coreless roll in a dispenser.

In yet another embodiment, the coreless roll may be dispensed from the periphery of the roll. The inner unperforated length of the absorbent tissue web is thereby dispensed at the end of the dispensing; as the last piece. The coreless roll may also be center-fed such that the inner unperforated length of the absorbent tissue web is dispensed at the start of dispensing. The unperforated length avoids a large chunk of wound material to be dispensed at once, i.e. the unperforated length promotes single unwinding and dispensing of every winding of the unperforated length.

In a further embodiment, the outermost winding, i.e. an outermost length of absorbent tissue web, of the coreless roll may also be unperforated. The outer tail of the web is attached to the periphery of the coreless roll by adhesives such that the outer tail does not flutter. However, in case the adhesive applied to attach the tail coincides with a perforation of the consecutive winding the web may break at the consecutive winding when the consumer attempt to detach the tail. An unperforated length of web at the outermost winding thereby minimize the risk of destroying consecutive windings and minimize the risk of unsatisfying dispensing.

“Releasably attached” is herein defined as webs adhering to each other if no separating force is applied, but separating from each other if a separating force is applied. In other words, the windings that are releasably attached are separated by normal peeling by hand. This does not include or affect the lamination of any ply bonding present within the web, i.e. the final individual sheets are intact after the windings of the coreless roll have been released from each other.

“Stabilized inner portion” is herein referred to the innermost portion of the roll, wherein the innermost windings do not collapse into the central axial hole. The innermost windings maintain their shape given to them by the mandrel to which they were wound during the manufacturing process. The shape of the central axial hole may be circular, hexagonal, star shaped or any other suitable shape in cross section.

“Stiffened” is herein defined as non-collapsing and maintaining its own shape. A couple of windings of the stiffened inner portion should by this definition be able to hold its own cylinder shape, whereas a couple of windings, wound as a cylinder, of the outer portion which is not stiffened collapse.

“High density” is herein defined as a coreless roll with a density of at least 235 kg/m³.

The various embodiments may be combined in various combination in accordance with this invention. Other advantages will become apparent from the hereinafter description of drawings and embodiments of the invention.

### BRIEF DESCRIPTION OF THE DRAWINGS

Various additional features and advantages of the invention will become more apparent to those of ordinary skill in the art upon review of the following detailed description of one or more illustrative embodiments taken in conjunction with the accompanying drawings. The accompanying drawings, which are incorporated in and constitute a part of this specification, illustrate one or more embodiments of the invention and, together with the general description given above and the detailed description given below, explain the one or more embodiments of the invention.

FIG. 1 is a perspective view of a coreless roll of absorbent tissue web according to one embodiment of the present invention.

FIG. 2 is a perspective view of the inner end of the coreless roll of absorbent tissue web of FIG. 1.

FIG. 3 is a plan view that schematically illustrates the length of a coreless roll of absorbent tissue web according to another embodiment of the present invention.

FIG. 4a is a top perspective view of a coreless roll according to another embodiment of the invention, the coreless roll having a stabilized inner portion.

FIG. 4b is a top perspective view of a coreless roll according to yet another embodiment of the invention, the coreless roll having an indentation at the first end.

FIG. 5 is a top plan view of a coreless roll according to yet a further embodiment of the invention, the coreless roll having a central axial bole defining a star shape.

### DETAILED DESCRIPTION

It is to be understood by one of ordinary skill in the art that the following is a description of exemplary embodiments only, and is not intended as limiting the broader aspects of the present disclosure.

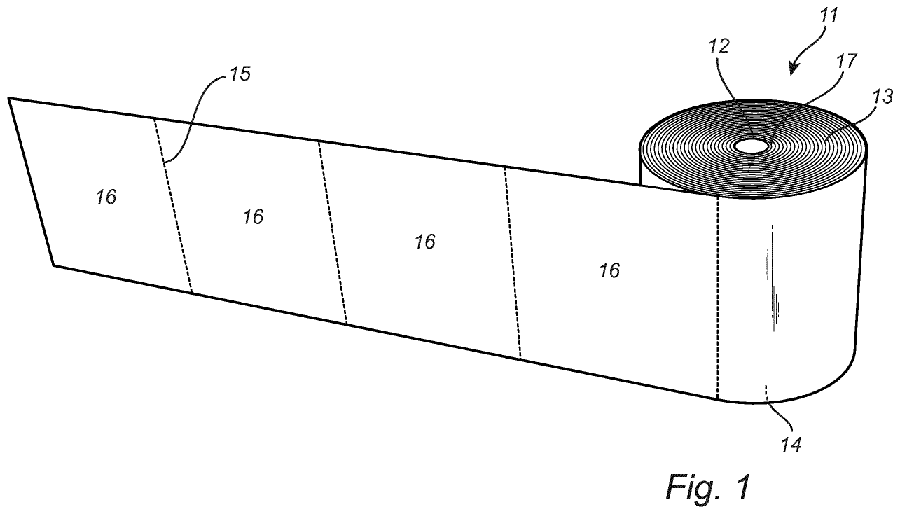

FIG. 1 shows a coreless roll 11 of absorbent tissue web, such as toilet paper, with a central axial hole 12. The coreless roll 11 have a cylindrical shape delimited axially by a first end 13 and a second end 14 opposite each other. The central axial hole 12 extends from the first end of the roll 13 to the second end of the roll 14 in a direction perpendicular to the length of the web L. The central axial hole of the coreless roll has a radius of about 6 mm+/−2 mm. The outer windings of the coreless roll 11, seen in FIG. 1, are provided with a plurality of perforation lines 15 across the width W of the web such as to define individual sheets 16. The perforation lines 15 are evenly distributed along the outer perforated length L2 of the absorbent tissue web. For the sake of illustration four individual sheets 16 are unrolled from the coreless roll in FIG. 1 but the individual sheets 16 are not separated.

The coreless roll 11 is a high density coreless roll with a density above 235 kg/m³, e.g., of about 270 kg/m³. The windings are tightly wound such as to create high density in the coreless roll 11. The central axial hole 12 is stabilized by the tightly wound windings.

The coreless roll comprises an inner portion 17 defined as the radial part arranged closest to the central axial hole. Each winding of the inner portion 17 is attached to the consecutive winding of the inner portion due to the tight winding. The attachments between the windings are releasable, such that the windings may be separated upon addition of a separating force as the coreless roll is unrolled by the consumer.

In this example, the inner portion 17 is stiffened by means, water, supplied to the web length corresponding to the inner portion 17 during manufacturing of the coreless roll 11. Each winding of the stiffened inner portion is at least partly bonded to the subsequent winding of the inner portion 17. The bonding is achieved by water added during the manufacturing process; which in the ready coreless roll 11 has evaporated and left the webs bonded.

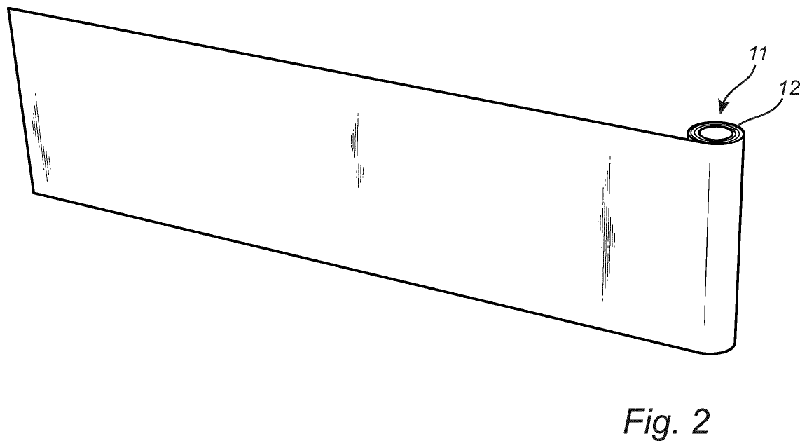

FIG. 2 shows the innermost part of the coreless roll 11 shown in FIG. 1. The absorbent tissue web at the end of the coreless roll 11 is unperforated i.e. the last length L2 of web is continuous. The inner windings are shown in FIG. 2, wherein the inner windings are releasably attached to each other at the inner portion.

The innermost, after winding, 2 meters of the absorbent tissue web has been sprayed with water in this example. The absorbent material of the inner windings seen in FIG. 2 are therefore effected by evaporated water. The water effects the paper by promoting adherence between the windings, and it also stiffens the material. A web that has been sprayed with water and dried does not have the same smoothness as a non-water-sprayed web. Moreover, the wetted absorbent tissue web creases more easily in the winding process. Creasing during the winding process also leads to subsequent windings attaching further to each other.

To avoid the risk of premature breaking in the inner windings due to the attachments; the absorbent tissue web at the inner windings of the inner portion 17 are unperforated, as seen in FIG. 2.

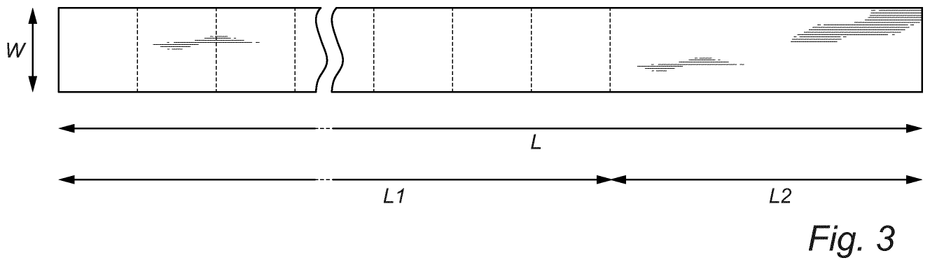

The length L of an absorbent tissue web according to some embodiments of the invention is seen in FIG. 3. It comprises a perforated length L1 and an unperforated length L2. The unperforated length L2 represents the length arranged closest to the central axial hole when being wound. The length L2 is longer than the length of each individual sheet 16.

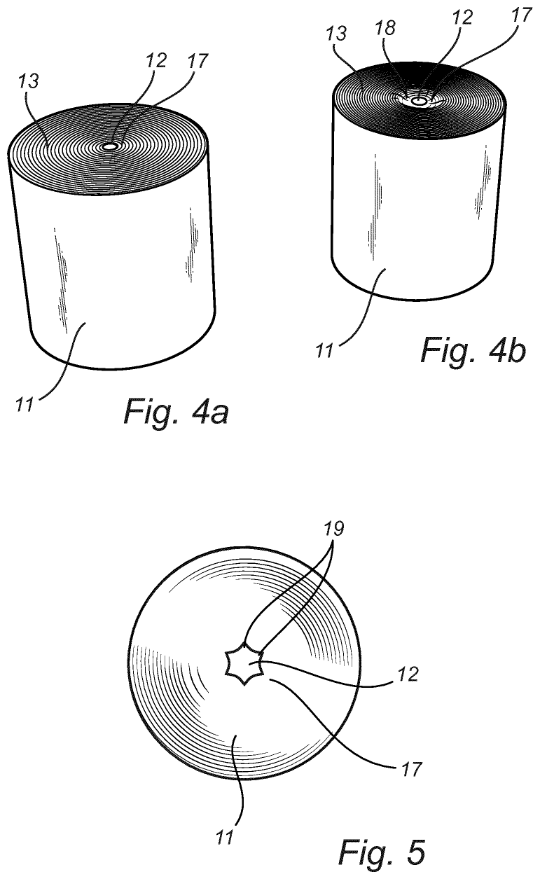

A coreless roll 11 with a stiffened inner portion 17 is seen in FIG. 4a. In this example, the inner portion 17 is stiffened by water, starch and plasticizer only. In the example shown in FIG. 4b, the coreless roll 11 of FIG. 4a is further provided with a cone shaped end indentation 18. The end indentation 18 affects the outer edges of the inner portion 17 and stabilizes them so that they do not collapse inwards. The end indentation (18) also contribute to the attachment of the windings of the inner portion 17. Part of the length constituting the inner portion 18 is unperforated. The unperforated length of absorbent tissue web provides strength such that the windings of the inner portion 17 may be unwound without breaking and creating “cigars” at the center.

FIG. 5 shows a coreless roll in which the central axial hole has a star shape, i.e. the coreless roll 11 is provided with inner indentations 19 along the inner surface of the central axial hole. The specific geometric shape is provided by the winding mandrel during manufacturing. The mandrel has in this case had a star shaped cross section. The inner indentations 19 stabilize the central axial hole 12 and contribute to the attachment between the windings of the inner portion 17. The inner windings indented by the mandrel thus has an improved attachment force compared to the windings of the outer portion of the coreless roll 11.

The invention is not limited to the examples of coreless rolls that have been depicted in the Figures. The application of the absorbent web product is broad and encompasses numerous away-from-home, domestic or sanitary applications, e.g. towels, kitchen towels, hand towels, toilet papers, wipes, facial tissues, bath tissues, napkins etc.

To this end, the embodiments described above are only descriptions of preferred embodiments of the present invention, and are not intended to limit the scope of the present invention. Various variations and modifications can be made to the technical solution of the present invention by those of ordinary skills in the art, without departing from the design and spirit of the present invention. The variations and modifications should all fall within the claim scope defined by the claims of the present invention.

What is claimed is:

1.-19. (canceled)

20. A coreless roll of absorbent tissue web, the absorbent tissue web having a length and a width, the roll defining a central axial hole that extends from a first end of the roll to a second end of the roll in a direction perpendicular to the length of the absorbent tissue web,
wherein the coreless roll has a density of at least 235 kg/m³,
wherein the absorbent tissue web comprises an outer perforated length of the absorbent tissue web that is wound in a periphery of the coreless roll, the outer perforated length comprising a plurality of perforation lines across the width of the absorbent tissue web such as to form individual sheets, and
wherein the absorbent tissue web of the coreless roll further comprises an inner unperforated length of the absorbent tissue web arranged and wound at an inner radial part of the coreless roll close to the central axial hole, such that the inner unperforated length of the absorbent tissue web is arranged closer to the central axial hole than the outer perforated length of the absorbent tissue web.

21. The coreless roll of absorbent tissue web of claim 20, wherein the inner unperforated length of the absorbent tissue web is longer than the length of the individual sheets.

22. The coreless roll of absorbent tissue web of claim 20, wherein the inner unperforated length of the absorbent tissue web has a length of 1 to 6 meters.

23. The coreless roll of absorbent tissue web of claim 20, wherein the inner unperforated length of the absorbent tissue web is arranged within an innermost 10 meters of the absorbent tissue web at the central axial hole.

24. The coreless roll of absorbent tissue web of claim 20, wherein the inner unperforated length of the absorbent tissue web represents inner windings arranged at an inner radius of the coreless roll such as to define the central axial hole.

25. The coreless roll of absorbent tissue web of claim 20, wherein the coreless roll comprises an inner portion arranged closest to the central axial hole, wherein windings of absorbent tissue web of the inner portion are releasably attached to each other.

26. The coreless roll of absorbent tissue web of claim 25, wherein the inner unperforated length of the absorbent tissue web is part of the inner portion.

27. The coreless roll of absorbent tissue web of claim 25, wherein the inner portion of the coreless roll is stiffened so as to maintain a shape of the central axial hole.

28. The coreless roll of absorbent tissue web of claim 27, wherein the coreless roll comprises an outer portion arranged closest to the periphery of the coreless roll, and wherein the stiffened inner portion is stiffer than the outer portion.

29. The coreless roll of absorbent tissue web of claim 25, wherein the inner portion is stiffened by application of a composition to the absorbent tissue web that is wound closest to the central axial hole.

30. The coreless roll of absorbent tissue web of claim 25, wherein the inner portion is stiffened by application of water to the absorbent tissue web that is wound closest to the central axial hole.

31. The coreless roll of absorbent tissue web of claim 25, wherein the inner portion is stiffened by application of adhesive applied to the absorbent tissue web that is wound closest to the central axial hole.

32. The coreless roll of absorbent tissue web of claim 25, wherein the inner portion is stiffened by application of starch applied to the absorbent tissue web that is wound closest to the central axial hole.

33. The coreless roll of absorbent tissue web of claim 25, wherein the first and/or second end of the coreless roll comprises an end indentation at the inner portion extending in an axial direction and arranged around the central axial hole.

34. The coreless roll of absorbent tissue web of claim 20, wherein the first and/or second end of the coreless roll has a shape of revolution with a cross section decreasing towards the central axial hole of the coreless roll.

35. The coreless roll of absorbent tissue web of claim 20, wherein the coreless roll comprises inner indentations at an inner surface of the central axial hole.

36. The coreless roll of absorbent tissue web of claim 20, wherein the outer perforated length of the absorbent tissue web defines a remaining strength of between 30% and 65% of an unperforated length of absorbent tissue web.

37. The coreless roll of absorbent tissue web of claim 20, wherein the individual sheets have a length between 8 cm and 40 cm.

38. A coreless roll of absorbent tissue web, the absorbent tissue web having a length and a width, the roll defining a central axial hole that extends from a first end of the roll to a second end of the roll in a direction perpendicular to the length of the absorbent tissue web,
wherein the coreless roll has a density of at least 235 kg/m³,
wherein the absorbent tissue web comprises an outer perforated length of the absorbent tissue web that is wound in a periphery of the coreless roll, the outer perforated length comprising a plurality of perforation lines across the width of the absorbent tissue web such as to form individual sheets,
wherein the absorbent tissue web of the coreless roll further comprises an inner unperforated length of the absorbent tissue web arranged and wound at an inner radial part of the coreless roll close to the central axial hole, such that the inner unperforated length of the absorbent tissue web is arranged closer to the central axial hole than the outer perforated length of the absorbent tissue web
wherein the inner unperforated length of the absorbent tissue web is longer than the length of the individual sheets,
wherein the inner unperforated length of the absorbent tissue web has a length of 1 to 6 meters,
wherein the inner unperforated length of the absorbent tissue web is arranged within an innermost 10 meters of the absorbent tissue web at the central axial hole,
wherein the inner unperforated length of the absorbent tissue web represents inner windings arranged at an inner radius of the coreless roll such as to define the central axial hole,
wherein the coreless roll comprises an inner portion arranged closest to the central axial hole, wherein windings of absorbent tissue web of the inner portion are releasably attached to each other,
wherein the inner unperforated length of the absorbent tissue web is part of the inner portion,
wherein the inner portion of the coreless roll is stiffened so as to maintain a shape of the central axial hole,
wherein the coreless roll comprises an outer portion arranged closest to the periphery of the coreless roll, and wherein the stiffened inner portion is stiffer than the outer portion,
wherein the inner portion is stiffened by at least one of the following: application of a composition to the absorbent tissue web that is wound closest to the central axial hole, application of water to the absorbent tissue web that is wound closest to the central axial hole, application of adhesive applied to the absorbent tissue web that is wound closest to the central axial hole, and application of starch applied to the absorbent tissue web that is wound closest to the central axial hole,
wherein the first and/or second end of the coreless roll comprises an end indentation at the inner portion extending in an axial direction and arranged around the central axial hole,
wherein the first and/or second end of the coreless roll has a shape of revolution with a cross section decreasing towards the central axial hole of the coreless roll,
wherein the coreless roll comprises inner indentations at an inner surface of the central axial hole,
wherein the outer perforated length of the absorbent tissue web defines a remaining strength of between 30% and 65% of an unperforated length of absorbent tissue web, and wherein the individual sheets have a length between 8 cm and 40 cm.
